(19) United States
(12) Patent Application Publication
(10) Pub. No.: US 2013/0090296 A1
(43) Pub. Date: Apr. 11, 2013

(54) INJECTABLE FORMULATION OF A MACROCYCLIC LACTONE AND LEVAMISOLE

(71) Applicants: Hyun Sun Cho, Sao Paulo (BR); Perterson Fabiano Lopes, Sao Brazil (BR)

(72) Inventors: Hyun Sun Cho, Sao Paulo (BR); Perterson Fabiano Lopes, Sao Brazil (BR)

(21) Appl. No.: 13/703,941

(22) PCT Filed: Jun. 23, 2011
(86) PCT No.: PCT/EP11/60535

§ 371 Date: Dec. 13, 2012

Related U.S. Application Data
(60) Provisional application No. 61/420,849, filed on Dec. 8, 2010.

(30) Foreign Application Priority Data
Jun. 24, 2010 (EP) 10167222.8

(51) Int. Cl. A61K 31/429; A61K 45/06; A61K 31/7048

(52) U.S. Cl. CPC A61K 31/429; A61K 31/7048; A61K 45/06
USPC 514/30; 514/368

(57) ABSTRACT

The present invention relates to formulations for controlling parasites, comprising a combination of macrocyclic lactone compound and levamisole wherein the levamisole is in particulate form suspended in a non-aqueous solvent

The present invention relates to injectable formulations for controlling parasites and the use of such formulations in the preparation of a medicament for controlling parasites.

Endoparasites commonly cause clinical disease in especially in livestock animals and have significant adverse economic effects on farming economies when present at subclinical levels. The most frequently encountered endoparasites are the group of worms referred to as nematodes. The nematodes are found in the intestinal tract, heart, lungs, blood vessels and other body tissues of animals and are a primary cause of anemia, weight loss and malnutrition in the infected animals. The nematodes most commonly found to be the infecting agents of ruminants include Haemonchus and Ostertagia generally found in abomasum; Cooperia, Trichostrongylus and Nematodirus generally found in the intestinal tract, and Dictyocaulus found in the lungs.

Treatment of animals to prevent infestation by any of the above-mentioned parasites, or to reduce or control the proliferation of these parasites in animals is thus important.

Meanwhile the problem has arisen that some parasites develop a resistance to antiparasitic drugs like ivermectin. The resistance occurs when a strain of a parasite is able to tolerate doses of an active ingredient that is efficacious against other populations of parasites of the same species. This characteristic is inheritable.

After the use of macrocyclic lactones (ML) for almost two decades in cattle in Brazil several reports on resistant endoparasites in sheep, cattle and goats were published.

The discovery of novel anti-parasitics with equal or better qualities than macrocyclic lactones seems to be a distant reality in the veterinary pharmaceutical industry.

Therefore, the chemical groups available nowadays must be used in a rational way, with a view to achieving high percentages of efficacy against endoparasites, especially in ruminants and delaying the occurrence of resistant strains.

Therefore a stable formulation for a combination of a macrocyclic lacone ad levamisole would be desirable However, such combinations have been difficult to formulate.

Accordingly, there is a need for a stable suspension formulation capable of including macrocyclic lactone compounds together with levamisole.

WO 00/74489 represents a recent attempt to formulate a combination avermectin/milbemycin and levamisole product.

Ashmont NZ 280085/280134 discloses injectable ML anthelmintic formulations (eg; abamectin or ivermectin) where an alcohol (such as benzyl alcohol-long since used as a preservative in injectable formulations) acts as a co-solvent with a vegetable oil vehicle (eg; soyabean oil, sesame oil or corn oil).

Associations of avermectines and levamisole have already been described before, in patent WO 00/061068 and in patent application WO 2004/009080.

### STATEMENT OF INVENTION

In one aspect the invention relates to a macrocyclic lactone solution formulation comprising levamisole in a particulate form in a non-aqueous solvent system.

In one embodiment the non-aqueous solvent system comprises oil and an organic solvent.

In one embodiment the oil comprises castor oil and a medium chain triglyceride or a mixture of medium chain triglycerides.

In one embodiment the organic solvent is dimethylacetamide.

In another aspect a stable formulation suitable for administration to animals including at least 2 actives wherein a first of the actives is a macrocyclic lactone and the second of said actives is levamisole, said actives being formulated in a stable suspension.

In another aspect the invention relates to stable formulations suitable for administration to animals including at least one active selected from the group comprising a macrocyclic lactone compound and levamisole and wherein levamisole being suspended in a non-aqueous solvent and is therefore in a particulate form in the formulation.

In another aspect the invention relates to such a formulation wherein the avermectin or milbemycin is present in the range of between 0.01-10% w/v.

In another aspect the invention relates to such a formulation wherein macrocyclic lactone compound is selected from the group comprising abamectin, doramectin, eprinomectin, ivermectin and moxidectin.

In another aspect the invention relates to such a formulation the levamisole is levamisole hydrochloride. In another aspect the invention relates to such a formulation wherein the levamisole is present in the range of between 1-40% w/v.

In another aspect the invention relates to such a formulation wherein the formulation additionally includes at least one further medicament selected from the group of anthelmintics, dietary supplements, vitamins, minerals and other beneficial agents.

In another aspect the invention relates to such a formulation the formulation is suitable for injectable administration.

In another aspect the invention relates to such a formulation, comprising: about 1.5-4% w/v ivermectin, about 15-18.8% w/v levamisole, and wherein the levamisole is particulate and the ivermectin is substantially in an non-aqueous solvent system comprising an oil and an organic solvent.

In another aspect the invention relates to such a formulation, wherein the oil comprises castor oil and a medium chain triglyceride or a mixture of medium chain triglycerides.

In another aspect the invention relates to such a formulation, wherein the organic solvent is dimethylacetamide.

In another aspect the invention relates to a method of treating or preventing infection of cattle with Cooperia or Ostertagia by administering a formulation as described above.

### DETAILED DESCRIPTION

The macrocyclic lactone solution formulation comprising levamisole in a particulate form in a non-aqueous solvent system is advantageous as it provides stable formulations including an avermectin or milbemycin in combination with levamisole.

The complicated nature of prior art formulations is due in part to the different formulation requirements of the actives. Avermectins and milbemycins being substantially insoluble in water, whereas levamisole is water soluble. In addition, levamisole has previously been found to require a pH of less than about 4 for stability while avermectins and milbemycin require a pH of about 6.6.

The formulations of the present invention exhibit desirable properties which are useful characteristics for the administration of relatively high concentrations of levamisole. The formulations are physically and chemically stable. In addition, as the formulation excludes water, the issue of incompatible pH requirements is alleviated. Enabling the two actives to stability co-exist in a single phase.

The formulations of the present invention must be stable to be of commercial use. In this specification, a commercially acceptable anthelmintic formulation is one which is stable at room temperature for a period of at least 6 months. In conditions of accelerated testing, at 40° C., this requires the potency of the actives within the formulation to remain within specified and acceptable limits for 3 months.

The macrocyclic lactones, i.e. the avermectin and milbemycin series of compounds are potent endo- and ectoparasitic agents. The compounds which belong to this series are either natural products or are semi-synthetic derivatives thereof. The structure of these two series of compounds are closely related and they both share a complex 1,6-membered macrocyclic lactone ring; the avermectins comprise a disaccharide substituent in the 1,3-position of the lactone ring, which the milbemycins do not. The macrocyclic lactones (avermectins and milbemycins) are products, or chemical derivatives thereof, of soil microorganisms belonging to the genus Streptomyces.

In a preferred embodiment of the invention, the macrocyclic lactones e.g. avermectins, milbemycins and derivatives thereof are selected from the group which includes but is not limited to, abamectin, doramectin, emamectin, eprinomectin, ivermectin, and selamectin (avermectin and derivatives thereof), milbemycin D, milbemycin oxime, lepimectin, and moxidectin (milbemycin and derivatives thereof) and mixtures thereof.

One particularly contemplated macrocyclic lactone parasiticide is ivermectin. Ivermectin is a semi-synthetic derivative of avermectin, and is generally produced as a mixture of at least 80% 22,23-dihydroavermectin B1ₐ and less than 20% 22,23-dihydroavermectin B1_{b}. Ivermectin is disclosed in U.S. Pat. No. 4,199,569. Ivermectin has been used as an antiparasitic agent to treat various parasitic diseases since the mid-1980's.

Other macrocyclic lactone parasiticides include, for example the following:

Abamectin This compound is, for example, identified as avermectin B1ₐ/B1_{b} in U.S. Pat. No. 4,310,519. Abamectin contains at least 80% of avermectin B1ₐ, and not more than 20% of avermectin B1_{b}.

Doramectin This compound is known as 25-cyclohexyl-avermectin B₁. Its structure and preparation are discussed in, for example, U.S. Pat. No. 5,089,480.

Emamectin This compound also is known as 4”-deoxy-4″-epi-methylaminoavermectin B₁. Its preparation is discussed in, for example, U.S. Pat. Nos. 5,288,710 and 5,399,717.

Eprinomectin This compound is known as 4″-epi-acetylamino-4″-deoxy-avermectin B₁. It was developed for use in all cattle classes and age groups.

Selamectin This compound also is known as 25-cyclohexyl-25-de (1-methylpropyl)-5-deoxy-22,23-dihydro-5-(hydroxyimino)-avermectin B1 monosaccharide.

Milbemycin and milbemycin oxime This compound also is known as B41. It is isolated from the fermentation broth of a Milbemycin-producing strain of Streptomyces. The microorganism, fermentation conditions, and isolation procedures are discussed in, for example, U.S. Pat. Nos. 3,950,360 and 3,984,564.

Moxidectin This compound is discussed in, for example, U.S. Pat. No. 4,916,154.

Lepimectin This is a chemically modified milbemycin macrolide (6R,13R,25R)-5-O-demethyl-28-deoxy-6,28-epoxy-13-[(Z)-[(methoxyimino)phenylacetyl]oxy]-25-methylmilbemycin B mixture with (6R,13R,25R)-5-O-demethyl-28-deoxy-6,28-epoxy-25-ethyl-13-[(Z)-[(methoxyimino) phenylacetyl]oxy] milbemycin B.

Preferably the avermectin or milbemycin is present in the range of between 0.01-10% w/v, 0.5-8% w/v, 1-6% w/v, 1.5-5% w/v, 1.6%-4% w/v.

Levamisole as used in this specification includes levamisole base, levamisole hydrochloride, levamisole phosphate together with other salts and forms. In one embodiment levamisole hydrochloride is used.

Preferably levamisole is present in the range of between 0.1-40% w/v, 10-35% w/v, 12-30% w/v, 15-25% w/v.

Good results were obtained with a formulation comprising 1.6-4% w/v ivermectin and 15.04-18.8% w/v levamisole hydrochloride.

By “particulate form” it is meant mobile, un-dissolved, solid matter suspended in a liquid. The liquid may be aqueous, oily, or both.

A solution is a mixture of two or more components that form a single phase that is homogeneous down to the molecular level.

A suspension consists of insoluble solid particles dispersed in a liquid medium, with the solid particles accounting for about 0.5% to about 30% of the suspension.

By “w/v” is meant weight/volume, i.e. “1% w/v” means 1 g in 100 ml of the formulation. “v/v” means volume per volume, and 1% v/v means 1 ml, in a total of 100 ml.

“Non-aqueous solvent system” means a solvent or a mixture of solvents that essentially consisting of liquid(s) other than water. The non-aqueous solvent system comprises at least one oil and at least one organic solvent.

Oils that can be used in pharmaceutical formulations are in general natural, e.g. vegetable, semi-synthetic or synthetic mono-, di- or tri glyceride. Vegetable oils are e.g. sesame oil, olive oil, cottonseed oil, castor oil, arachis oil, coconut oil.

In a preferred embodiment the pharmaceutical formulation according to the invention is characterised in that the oil is castor oil together with a low viscosity medium chain triglyceride or a mixture of medium chain triglycerides.

Castor oil (ricinus oil, oleum ricini, ricinoleum, tangantangan) is a triglyceride of fatty acids. The fatty acid composition is approximately ricinoleic acid (87%); oleic acid (7%), linoleic acid (3%); palmitic acid (2%); stearic acid (1%) and trace amounts of dihydroxy stearic acid. Castor oil is the fixed oil obtained by cold-expression of the seeds of Ricinus communis Linnè (Fam. Euphorbiaceae). (Rowe R C et al: Handbook of Pharmaceutical Excipients, London, Pharmaceutical Press, GB, page 104-105).

Medium chain triglycerides (MCT) have fatty acid chains of 6-12 carbon atoms and for the medically refined grades of MCT oil each chain has 8-10 carbon atoms.

The MCT oil may comprise either triglycerides of the C8-C10 fatty acids, or propylene glycoldiesters of these fatty acids or a mixture of both triglycerides and propylene glycol diesters. Preferably these C8-C10 fatty acids are fully saturated, such as n-caprylic and n-capric acids. These are conveniently prepared by the commercial fractionating of naturally occurring vegetable (e.g. coconut) oil to give mainly C8-10 fatty acids followed by esterification of these acids with a chosen alcohol.

Fractionated vegetable oil having the desired composition is commercially available. Proprietary examples of such oils are Miglyol® 812 as capric/caprylic triglycerides and Miglyol® 840 as propylene glycol dicaprylate/caprate.

Equivalents of these oils are for example: Aldo® MCT KFG, Aldo® TC, Calgene CC-33, Calgene CC-33-F, Calgene CC-33-L, Calgene CC-33-S, Captex® 300, Captex® 355, Crodamol GTCC, Estasan GT 8-40 3578, Estasan GT 8-60 3575, Estasan GT 8-60 3580, Estasan GT 8-65 3577, Estasan GT 8-65 3581, Estasan GT 8-70 3579, Labrafac® LIPO, Labrafac® lipophile WL 1349, Lexol® GT-855, Lexol® GT-865, Miglyol® 810, Miglyol® 812, Myritol® 312, Myritol® 318, Neobee® 1053, Neobee® M-5, Neobee® O, Pelemol® CCT, Standamul® 318, Standamul® 7105 and Calgene CC-22, Calgene CC-22-S, Captex® 200, Lexol® PG-865, Miglyol® 840, Myritol® PC, Neobee® 1054, Neobee® M-20, Pelemol® PDD, Standamul® 302.

The organic solvent must be pharmaceutically acceptable and is preferably selected from Dimethylacetamide (N,N-dimethylacetamide) and DMSO and mixtures thereof .

The formulation according to the current invention may further comprise additional pharmaceutical excipients known in the art. Such pharmaceutical excipients are e.g. described in “Gennaro, Remington: The Science and Practice of Pharmacy”, (20. Edition, 2000), incorporated by reference herein. Such ingredients include preservatives, chelating agents, antioxidants and stabilizers.

Suitable minerals and vitamins that can be included in the veterinary formulation are known. Exemplary preservatives include methyl p-hydroxybenzoate (methylparaben) and propyl p-hydroxybenzoate (propylparaben). Exemplary chelating agents include edetate sodium. Exemplary antioxidants include butylated hydroxyanisole and sodium monothioglycerol.

Preferred formulations of the present invention comprise:
Component 1—Macrocyclic lactone compound
Component 2—An oil (vegetable or mineral)
Component 3—levamisole
Component 4—organic solvent
Component 5A, 5B, etc.—Additional active(s) of which at least one is preferably an anthelmintic active. This or these can be either dissolved in the water phase (such as Levamisole) or suspended in the water phase (such as Albendazole) if insoluble, or both (e.g. Levamisole and Albendazole).

Additional components can be added to this basic formulation.
Addition 1—Minerals/Vitamins
Addition 2—Preservatives.

Preferred components and their concentration ranges and examples are set out in Table 1.

TABLE 1

| | Function | Examples | % W/V |
|---|---|---|---|
| Component 1 | Macrocyclic | Abamectin or ivermectin | 0.1-10% |
| | lactone | | |
| Component 2 | Vegetable | castor oil, caprilic/capric | 1-60% |
| | Oil Mineral | acid triglyceride | 1-To Volume |
| | Oil | | |
| Component 3 | Levamisole | levamisole hydrochloride | 0.1-40% |
| Component 4 | organic | Dimethylacetamide | 5-20% |
| | solvent | | |
| Component | Additional | | 0-25% |
| 5A, 5B, etc. | anthelmintic | | |
| Addition 1 | Minerals | Mineral salts or chelates | 0-5% |
| Addition 2 | Preservatives | Benzoic Acid, Potassium | 0-1% |
| | | Sorbate and Parabens, | |
| | | Benzyl Alcohol, | |
| | | methylhydroxytoluen | |

In a preferred embodiment the formulations according to this invention are used to treat a helminth infection, such as an infection caused by one or more helminths selected from the group consisting of Ancylostoma spp.; Anecator spp.; Ascaridia spp.; Ascaris spp.; Brugia spp.; Bunostomum spp.; Capillaria spp.; Chabertia spp.; Cooperia spp.; Cyathostomum spp.; Cylicocyclus spp.; Cylicodontophorus spp.; Cylicostephanus spp.; Craterostomum spp.; Dictyocaulus spp.; Dipetalonema spp; Dirofilaria spp.; Dracunculus spp.; Enterobius spp.; Filaroides spp.; Habronema spp.; Haemonchus spp.; Heterakis spp.; Hyostrongylus spp.; Metastrongylus spp.; Meullerius spp. Necator spp.; Nematodirus spp.; Nippostrongylus spp.; Oesophagostomum spp.; Onchocerca spp.; Ostertagia spp.; Oxyuris spp.; Parascaris spp.; Stephanurus spp.; Strongylus spp.; Syngamus spp.; Toxocara spp.; Strongyloides spp.; Teladorsagia spp.; Toxascaris spp.; Trichinella spp.; Trichuris spp.; Trichostrongylus spp.; Triodontophorous spp.; Uncinaria spp., and/or Wuchereria spp.;

In cattle the major production limiting parasite species is Ostertagia. spp. Another important cattle parasite is Cooperia. spp.

The formulations according to this invention may be used to treat animals, including humans and non-human animals, especially non-human mammals. Such non-human mammals include, for example, livestock mammals (e.g., swine, livestock ruminants like bovines, sheep, goats, etc.), laboratory mammals (e.g., mice, rats, jirds, etc.), companion mammals (e.g., dogs, cats, equines, etc.), and wild and zoo mammals (e.g., buffalo, deer, etc.). It is contemplated that the compounds according to this invention also are suitable to treat non-mammals, such as poultry (e.g., turkeys, chickens, ducks, etc.) and fish (e.g., salmon, trout, koi, etc.).

In some embodiments, one or more, preferably one compound according to this invention is used to treat an infection by a helminth, such as a nematode, cestode or trematode, preferably a nematode (such as Haemonchus contortus), that is resistant to one or more other anthelmintic agents. In some embodiments, the compound according to this invention is active against a helminth, such as a nematode, cestode or trematode, preferably a nematode such as Haemonchus contortus, that is resistant to one or more of the following anthelmintics: an avermectin (e.g., ivermectin, selamectin, doramectin, abamectin, and eprinomectin); a milbemycin (moxidectin and milbemycin oxime); a pro-benzimidazole (e.g., febantel, netobimin, and thiophanate); a benzimidazole derivative, such as a thiazole benzimidazole derivative (e.g., thiabendazole and cambendazole) or a carbamate benzimidazole derivative (e.g., fenbendazole, albendazole (oxide), mebendazole, oxfendazole, parbendazole, oxibendazole, flubendazole, and triclabendazole); an imidazothiazole (e.g., levamisole and tetramisole); a tetrahydropyrimidine (morantel and pyrantel), an organophosphate (e.g., trichlorphon, haloxon, dichlorvos, and naphthalophos); a salicylanilide (e.g., closantel, oxyclozanide, rafoxanide, and niclosamide); a nitrophenolic compound (e.g., nitroxynil and nitroscanate); benzoenedisulphonamide (e.g., clorsulon); a pyrazinaisoquinoline (e.g., praziquantel and epsiprantel); a heterocyclic compound (e.g., piperazine, diethylcarbamazine, dichlorophen, and phenothiazine); an arsenical (e.g., thiacetarsamide, melorsamine, and arsenamide); cyclooctadepsipeptide (e.g., emodepside); and a paraherquamide.

The formulation according to the invention can be applied to an animal in general by all application forms known in the art. Generally the administration to the animal is done orally or parenterally. While the pharmaceutical formulation according to the current invention is preferably administered parenterally, e.g. by intraveneous, intramuscular or subcutaneous injection, treatment via alternative routes is also possible.

The following examples are provided as examples only and are in no way intended to limit the spirit or scope of the invention.

Example Formulations

The formulations of the present invention were prepared as follows:

```
1. Transfer the following compounds to a capable vessel fitted with an
agitator and an extractor system in the following order:
                        N,N-dimethylacetamide
                        ivermectin
2. Stir until dissolved.
3. Transfer the following compounds to another capable vessel fitted with
an agitator and an extractor system in the following order:
                        Castor oil
                        Caprilic/capric triglyceride
                        Butylated hydroxytoluene
4. Stir until dissolved. Add the ivermectin solution and stir until complete
homogenization.
5. Filter the solution through a sterilizing membrane to a sterile vessel with
agitator and under laminar flow and add: Levamisole hydrochloride
6. Stir until complete homogenization and pass through cycling mill to get
homogenous suspension of levamisole.
```

The stability and efficacy of such formulations was investigated.

Formulation (F1)
Levamisol Hydrochloride—18.8%
Ivermectin—2%
Dimetylacetamide—10%
Butylhydroxytoluen—0.02%
Castor oil—50.4%
Crodamol GTCC—up to 100 mL

Formulation (F2)
Levamisol hydrochloride—15.04%
Ivermectin—1.6%
Dimetylacetamide—10%
Butylhydroxytoluen—0.02%
Castor oil—50.4%
Crodamol GTCC—up to 100 mL

Formulation (F3)
Levamisol hydrochloride—18.8%
Ivermectin—4%
Dimetylacetamide—10%
Butylhydroxytoluen—0.02%
Castor oil—50.4%
Crodamol GTCC—up to 100 mL

Formulation (F4)
Levamisol hydrochloride—15.04%
Ivermectin—3.2%
Dimetylacetamide—10%
Butylhydroxytoluen—0.02%
Castor oil—50.4%
Crodamol GTCC—up to 100 mL

TABLE 2
Stability data (Formulation F3)
Accelareted Stability Studies (40° C./75% R Humidity)

| | intial | | 1st month | | 2nd month | | 3rd month | | 6 month | |
|---|---|---|---|---|---|---|---|---|---|---|
| | levamisole | | levamisole | | levamisole | | levamisole | | levamisole | iver- |
| | hydrochloride | ivermectin | hydrochloride | ivermectin | hydrochloride | ivermectin | hydrochloride | ivermectin | hydrochloride | mectin |
| Batch | (%) | (%) | (%) | (%) | (%) | (%) | (%) | (%) | (%) | (%) |
| 001/09 | 18.48 | 1.99 | 18.76 | 2.01 | 19.09 | 1.98 | 19.91 | 2.10 | 18.82 | 2.00 |
| 002/09 | 15.05 | 1.56 | 14.91 | 1.54 | 15.47 | 1.59 | 15.21 | 1.59 | 15.37 | 1.63 |
| 003/09 | 14.73 | 3.11 | 14.12 | 3.18 | 14.67 | 3.08 | 14.46 | 3.16 | 14.09 | 3.12 |
| 004/09 | 19.04 | 3.80 | 18.29 | 3.91 | 18.77 | 3.67 | 18.80 | 4.01 | 18.71 | 3.74 |

TABLE 3
Nematode efficacy data Formulation (F3 in Group IV and V)) Percentages of nematodes control in cattle
Geometric means. CPPAR/Jaboticabal/UNESP, Jaboticabal-SP, Brazil

| | Groups/Average number of nematodes detached from experimental animals | | | | | | | | | | | | |
|---|---|---|---|---|---|---|---|---|---|---|---|---|---|
| | (EPG) | | | | | | | | | | | | |
| | | | | GIV: CPI 004 | GV: CPI 004 | | | | | | | | |
| | | | | L03(5) | L01(6) | | | | | | | | |
| | | | | Levamisol | Levamisol | | | | | | | | |
| | | GII: | | Hydrochloride | Hydrochloride | | | | | | | | |
| | | Ivermectin | | 15.05% + | 18.8% + | GVI: | GVII: | | | | | | |
| Days | | 3.15% | GIII: | Ivermectin | Ivermectin | Ivermectin | Ivermectin | | | | | | |
| After - | GI: | (Ivomec | levamisole | 3.2% (dose | 4% (dose | 1% (Cepa | 1% (Cepa | Percentages of Efficiency (%) | | | | | |
| Treatment | Control | Gold) | 18.8% | 1 mL/40 Kg) | 1 mL/50 Kg) | Resistent) | Sensitive) | GII | GIII | GIV | GV | GVI | GVII |
| Day 0 | 1097.03 | 1138.95 | 1256.68 | 1297.92 | 1353.01 | 1284.54 | 1310.98 | — | — | — | — | — | — |
| 1 | 1097.19 | 623.98 | 16.15 | 1.96 | 2.85 | 1195.28 | 909.18 | 43.13 | 98.53 | 99.82 | 99.74 | 0.00 | 17.14 |
| 3 | 873.64 | 995.71 | 11.55 | 7.74 | 0.72 | 1781.44 | 393.25 | 0.00 | 98.68 | 99.11 | 99.92 | 0.00 | 54.99 |
| 5 | 824.17 | 1305.06 | 16.15 | 6.64 | 1.96 | 1454.11 | 54.75 | 0.00 | 98.04 | 99.19 | 99.76 | 0.00 | 93.36 |
| 7 | 1240.65 | 1001.27 | 21.96 | 7.17 | 22.35 | 4314.56 | 98.85 | 19.29 | 98.23 | 99.42 | 98.20 | 0.00 | 92.03 |

1. A macrocyclic lactone solution formulation comprising levamisole in a particulate form in a non-aqueous solvent system.

2. The formulation as claimed in claim 1, wherein the non-aqueous solvent system comprises oil and an organic solvent.

3. The formulation as claimed in claim 1, wherein the oil comprises castor oil and a medium chain triglyceride or a mixture of medium chain triglycerides.

4. The formulation as claimed in claim 1, wherein the organic solvent is dimethylacetamide.

5. The formulation as claimed in claim 1, wherein the macrocyclic lactone compound is present in the range of between 0.01-10% w/v.

6. The formulation as claimed in claim 1, wherein macrocyclic lactone compound is selected from the group comprising abamectin, doramectin, eprinomectin, ivermectin and moxidectin.

7. The formulation as claimed in claim 1, wherein the levamisole is levamisole hydrochloride.

8. The formulation as claimed in claim 1, wherein the levamisole is present in the range of between 1-40% w/v.

9. The formulation as claimed in claim 1, wherein the formulation additionally includes at least one further medicament selected from the group of anthelmintics, dietary supplements, vitamins, minerals.

10. The formulation as claimed in claim 1, wherein the formulation is suitable for injectable administration.

11. The formulation as claimed in claim 1, comprising:
about 1.5-4% w/v ivermectin,
about 15-18.8% w/v levamisole, and
wherein the levamisole is particulate and the ivermectin is substantially in an non-aqueous solvent system comprising an oil and an organic solvent.

12. The formulation as claimed in claim 11, wherein the oil comprises castor oil and a medium chain triglyceride or a mixture of medium chain triglycerides.

13. The formulation as claimed in claim 11, wherein the organic solvent is dimethylacetamide.

14. A method of treating or preventing helminth infection of cattle with Cooperia or Ostertagia spp. comprising administering to the cattle the formulation of claim 1.

15. (canceled)
